(19) United States
(12) Patent Application Publication
(10) Pub. No.: US 2004/0191889 A1
(43) Pub. Date: Sep. 30, 2004

(54) See through testing device

(76) Inventor: Lalit Mahajan, New Delhi (IN)

Correspondence Address: LADAS & PARRY, 26 WEST 61ST STREET, NEW YORK, NY, 10023, US

(21) Appl. No.: 10/480,414

(22) PCT Filed: Jun. 15, 2001
(86) PCT No.: PCT/IN01/00116

(51) Int. Cl. C12Q 1/56; C12M 1/34

(52) U.S. Cl. 435/287.1; 435/13

(57) ABSTRACT

A “see-through” testing device used for detecting analytes in biological fluid comprising an opaque bottom receptacle (1), a transparent top (18) with a sample cup (17) and a collar (13).

The subject invention relates to a “See-through” testing device used for the purpose of detecting analyte in Blood, Serum, Plasma, other human body fluids.

The device is used in a immunochromatographic assay. In the technical parlance tests are known as “Lateral Flow Rapid Test” for the detection of analytes.

### BACKGROUND OF THE INVENTION

In the available devices as used conventionally, the devices used for the purpose of detection of analyte in blood, serum, plasma and other body fluids, the devices were made up of opaque plastic wherein only the sample cup and the reaction window was visible. The main drawback associated with these devices is that it is not possible to visualize the progress of the reaction chain, hence the set back in the reaction chain could not be analyzed.

Additionally in the conventionally available devices, a window is provided for the observation of the sample analysis reading. This window results in causing the evaporation of the sample during hot and dry summers, thus occasionally stopping the reaction in the middle of the window. Due to rapid rate of evaporation because of very low humidity and high ambient temperature, the conventionally available devices generally results in providing the wrong results.

Since the samples are highly infectious containing antibodies or antigens, which can prove fatal, there is always a possibility of accidental touch by the user. As all the conventional test devices incorporate an exposed window.

The sample cup as available conventionally, were not tailored to conform to the size of the test strip, thus sometimes leakage of the sample was evident.

To overcome this difficulty the “see-through” testing device of the subject invention has been devised, which is not only user friendly but where it is also possible to supervise the reaction steps at every stage.

The “See-through” testing device of the subject invention enables the user of this device to watch the progress of the reaction at each step till the completion of the reaction.

The “see through” testing device of the subject invention is totally enclosed without having any open window, thus there is no possibility of the user of the device even remotely to touch the infected sample and moreover after completion of the test, the discarding of the complete device containing infectious material is easier and safer.

Moreover, sometimes due to the transportation or wrong storage of the device, the sample gets deteriorated or spilled over in conventional devices as it is not possible to detect such happenings because of the opacity and non-transparency of the device.

In the subject device, the conjugate will not flow in the reaction even it is not properly stored or transported and any problem in the conjugate may be easily visualized by the user.

Moreover, due to the transparency of the device, any leakage or spillage inside the device at the time of addition of sample on the test strip will also be identified, thus the result of the test will not be affected.

The see through testing device of subject application comprises mainly of the top transparent component and the Bottom receptacle.

The top component is made up of the material selected from Polystyrene, Polycarbonate or the like suitable transparent material.

The bottom receptacle is made up of the material selected from Polypropylene, Polyethylene, PVC or the like suitable opaque material.

The bottom receptacle comprises the dimensional configuration of the top component to provide air-tight compartment.

Accordingly, the present invention relates to a “See-through” testing device used for the purpose of detecting analyte in Blood, Serum, Plasma and other human body fluids comprising an opaque bottom receptacle and a transparent top component having a collar snugly fitting into the cavity provided around the said opaque bottom receptacle, conforming dimensional configuration with said bottom receptacle to provide a air-tight compartment, characterized in that the top component comprises a converging sample cup provided near one end of the said top component having its bottom width three fourth to the width of the test strip and having a depth providing hair gap between the sample pad of the test strip and the bottom of the sample cup; means for controlling the desired lateral flow of conjugate along the test strip; means for keeping the sink pad of the test strip in its desired position; means for maintaining the temperature inside the device in conformity with outside temperature to avoid formation of fogginess or mist, and the bottom receptacle comprises two pair of tracks provided at both the ends of the inner side of the bottom receptacle to hold the sample pad and sink pad of the test strip in position; a test line provided near the center of the bottom receptacle; and a Control Line provided near the sink pad of the test strip.

The means for controlling the proper flow of conjugate on the test strip is a bar positioned near the sample cup. The means for keeping the sink pad of the test strip on its proper desired position is a bar positioned near the sink pad of the test strip. These bars are positioned on the inner side of said top component. The means for maintaining the temperature inside the device in conformity with outside temperature to avoid formation of fogginess or mist are plurality of ventilators provided on the outer side of the top component. These ventilators are pair of circular holes or slits provided at the center of the top component of the said device having the diameter of 10 mm².

The top component and bottom receptacle are rectangular in shape.

The sample cup is rectangular in shape having the width at its bottom of 2 mm and depth of 2.5 mm to 3.0 mm.

The length of said tracks adjacent the sink pad of the test strip is more then the length of the tracks adjacent the said sample pad of the test strip.

The subject application may better be understood with reference to the accompanying drawings, which are for illustrative purposes and the same should not be construed to restrict the scope of the application.

### BRIEF DESCRIPTION OF THE ACCOMPANYING DRAWINGS

FIG. 1: relates to a conventionally available testing device;

FIG. 2: relates to the testing device of subject invention exhibiting positioning of the test strip in the base of said bottom receptacle;

FIG. 3: relates to the positing of pair of tracks;

FIG. 4: relates to the top component of the said device;

FIG. 5: relates to the sample cup of the top component of the device.

### DETAILED DESCRIPTION OF THE INVENTION

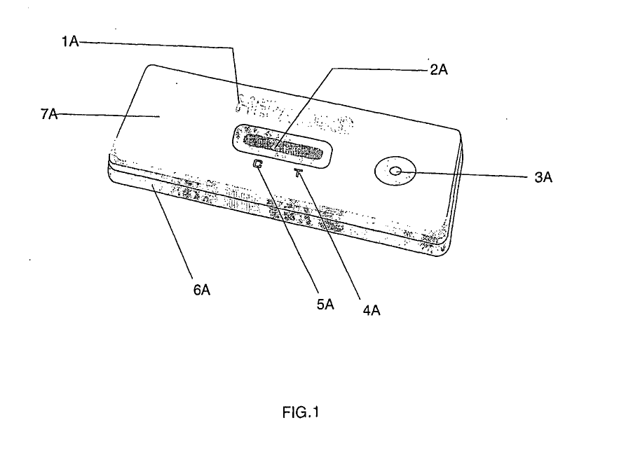

As shown in FIG. 1 the conventionally available testing device is completely opaque having a window (2A) to see the end result and a circular opening (3A) for the receipt of sample. This window results in causing the evaporation of the sample during hot and dry summers, thus occasionally stopping the reaction in the middle of the window due to rapid rate of evaporation. Moreover, there is always a possibility of accidental touch by the user. As all the conventional test devices incorporate an exposed window. The opening (3A) for the receipt of the sample is such, which occasionally leads to the spilling over of sample.

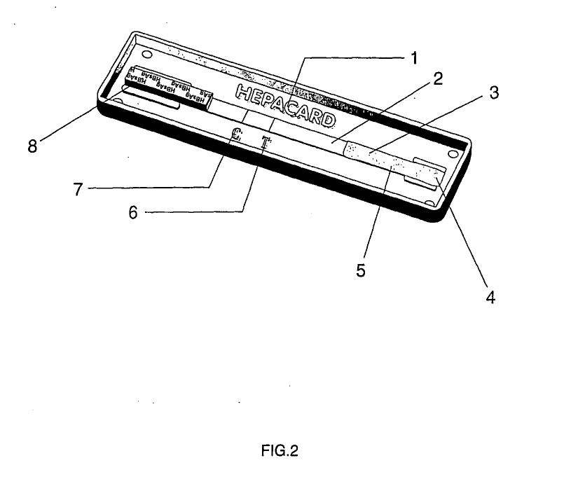

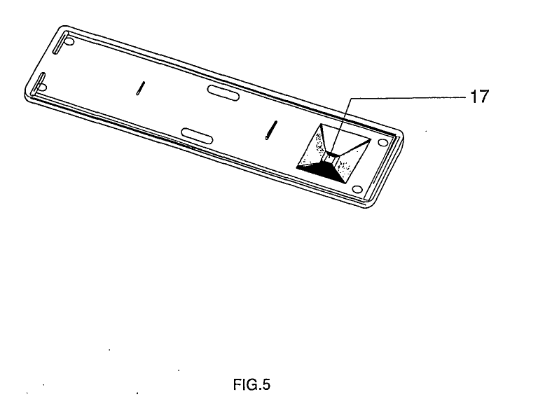

See-through testing device of the subject invention as shown in FIGS. 2-5 are air tightly enclosed; there is no evaporation of the sample from the test strip surface. The amount of sample required is just 2 drops (70 Microliters), which is ideal in case of pediatric, infant, geriatrics samples where very small amount of blood or fluid is available for testing.

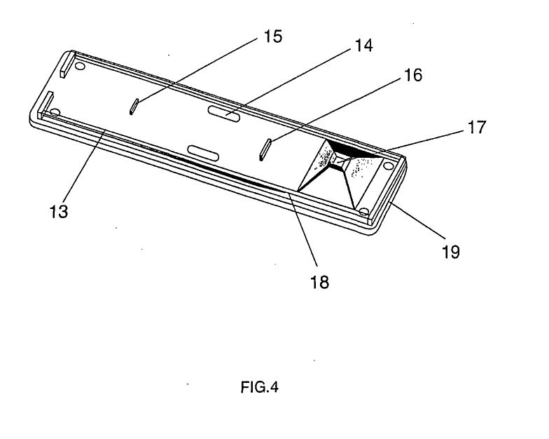

The transparent top component (18) as shown in FIG. 4, of the subject device is rectangular in shape and is provided with a collar (13) snugly fitting into the cavity provided around the rectangular opaque bottom (1) receptacle to make the device completely air-tight.

The transparent top component (18) comprises a sample cup (17) and pair of bars (15 & 16) provided on the inside of the said transparent top component (18).

The sample cup (17) is rectangular at the top and rectangular at the bottom side having the covering sides so designed that it can accommodate 2 drops (70 Microliters) of the sample under test. The bottom outlet of the said sample cup (17) is also rectangular in shape having diameter of about 2 mm .

The test strip used is having a width of 3 mm to allow the bottom of sample cup (17) which is of 2 mm dimension to rest with a hair gap within the 3 mm width of the strip.

Depth of the said sample cup is so optimized that it has a hair gap between the bottom of sample cup (17) and top of sample pad(4), thus avoiding any spillage of the sample. Other side of the rectangular cup sample (17) bottom depth is approx. 2.5 mm to 3.0 mm.

A pair of bars (15 & 16) are provided on the inside of the said top component (17) to ensure the proper flow of the conjugate.

The depth of the bar (15 & 16) is very crucial as if the bar is less in depth, conjugate pad (4) will not get pressed and it will be open thereby disturbing the flow of the conjugate making it very irregular.

If depth of this Bar is excessive, then it will press the conjugate pad tightly and will not allow conjugate to flow along the test strip, thus resulting in giving the invalid results.

The bar (15) near the sink pad (8) is so designed to keep the sink pad (8) in position. The depth of this bar (15) is so adjusted so there is a hair gap between the sink pad and the lower surface of the said bar (15) to keep the sink pad (8) in the desired position.

In “see-through” testing device, the top component (18) has a very unique feature of the temperature compensation inside the device so that the temperature inside the device is more or less in conformity with the outside temperature. This has been achieved by providing ventilators (14) in the form of corresponding holes or slit (14) having a total area of approximately 10 mm². These opening provide ventilation between the inside area and outside area so that no fogginess or mist appears on the inside of the “see-through” device while performing the test.

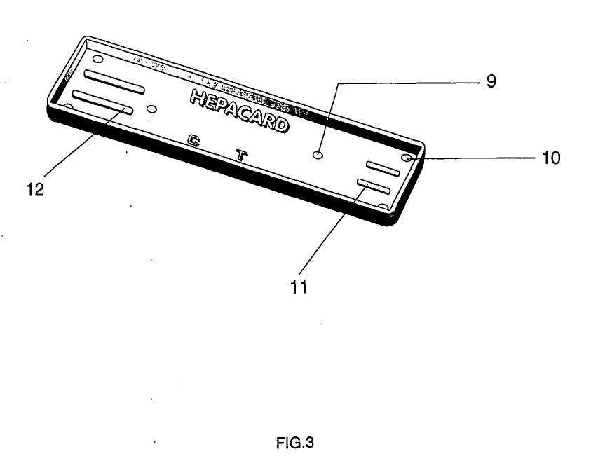

In the bottom receptacle (19) of the subject “see-through” testing device HEPACARD and the word “C” & “T” inscribed on the bottom side as shown in FIGS. 2 and 3. There are two tracks (11 and 12) as shown in FIG. 3, providing on the inside surface on both the sides of the said bottom receptacle (19) to hold the test strip.

The length of the track (11) adjacent to sample cup (17) is 7.5 millimeters so that it can hold the sample cup (17) in proper balance. The height of this track (11) is optimized so that bottom of the Sample cup (17) is placed accurately on the sample pad (4), guiding the sample cup (17) in proper position and for the smooth flow of the sample under test.

Height of this track (11) is so adjusted that right amount of the sample is released gradually on the strip from the said sample cup(17). If the height is less; then the sample cup (17) will rest on the strip (4), which will press the strip thus blocking the flow of the sample. If the height of the track is more, a gap between the sample cup (17) and the strip will be there causing the sample cup (17) to rest on the track (11 & 12) and not on the sample pad resulting in spilling over the sample.

The track (12) on the other side is to hold the sink pad in position of the testing strip (4).

When the sample is added in the sample cup (17) for the test, the sample flows forward and then combines with the conjugate provided in the test strip containing antibody or antigen to form a complex. The conjugate is a corresponding antibody or antigen linked gold conjugate. This complex thus formed consists of analyte-antibody or antigen-gold conjugate complex. This complex moves forward along the test strip attaching with the corresponding antibodies/antigens on the Test Line “T” (6). The complex so formed is antibody/antigen (Test line)—analyte—gold conjugate antibody/antigen complex. This complex appears as a purple line on the test strip (4) exhibiting the positivity of the sample.

The remaining sample continues to move further on the test strip (4) towards the Control line “C” (7). Control line (7) contains antibody namely Anti Human IgG antibody. Some of the complex sets attached at the Control Line (7), again giving a purple colour. Thereby exhibiting the validity of the device and the procedure.

In case this line does not appear, the test is considered invalid. Therefore in case of a positive sample, there are two purple lines formed on the Test Strip(4). One line at the test region “T” (6) and the second line at the control region “C”(7)

The test strip (4) as shown ion FIG. 2 comprises sample pad (5), conjugate pad containing conjugate (3), test line (6), control line (7) and sink pad (8).

In case the sample does not contain analyte, the sample flows as usual with conjugate and moves further, laterally. The complex moving forward is antibody/antigen—Gold conjugate complex. Since the sample does not contain any analyte, the conjugate moving forward on the test strip (4) will not have any binding on the test line “T” (6) region and no colour will appear. However the complex gets bound to the anti human IgG antibodies on the Control line “C” (7). So there will be only one purple/pink colour line on the “C” (7) control region indicating that the test is valid. There will be no purple line in the Test Region “T” (6). In case no purple/pink line appears on the Control region, “C”(6), the test is considered invalid.

The result for the present analyte in the sample is detected at a very short interval, maximum time being twenty minutes.

The subject application is a statement of invention, wherein various alterations and modifications are possible without deviating from the scope of the claimed invention, hence the same should not be construed to restrict the scope of the invention.

1. (Previously presented) A “See-through” testing device used for the purpose of detecting analyte in Blood, Serum, Plasma and other human body fluids comprising an opaque bottom receptacle and a transparent top component having a collar snugly fitting into the cavity provided around the said opaque bottom receptacle, confirming dimensional configuration with said bottom receptacle to provide a air-tight compartment, characterized in that the top component comprises a converging sample cup provided near one end of the said top component having its bottom width three fourth to the width of the test strip and having a depth providing hair gap between the sample pad of the test strip and the bottom of the sample cup; means for controlling the desired lateral flow of conjugate along the test strip; means for keeping the sink pad of the test strip in its desired position; a plurality of ventilators provided on the outer side of transparent top component to maintain the temperature inside the device in conformity with the outside temperature to avoid formation of fogginess or mist, and the bottom receptacle comprises two pair of tracks provided at both the ends of the inner side of the bottom receptacle to hold the sample pad and sink pad of the test strip in position; a test line provided near the center of the bottom receptacle; and a Control Line provided near the sink pad of the test strip.

2. (Previously presented) A see through testing device as claimed in claim 1, wherein the top component is made up of the material selected from Polystyrene, Polycarbonate or the like suitable transparent material.

3. (Previously presented) A see through testing device as claimed in claim 1, wherein the bottom receptacle is made up of the material selected from Polypropylene, Polyethylene, PVC or the like suitable opaque material.

4. (Previously presented) A see through testing device as claimed in claim 1, wherein the means for controlling the proper flow of conjugate on the test strip is a bar positioned near the sample cup.

5. (Previously presented) A see through testing device as claimed in claim 1, wherein the means for keeping the sink pad of the test strip on its proper desired position is a bar positioned near the sink pad of the test strip.

6. (Currently amended) A see through testing device as claimed in claim 4 and 5, wherein the bars are positioned on the inner side of the top component.

7. (Previously presented) A see through testing device as claimed in claim 1, wherein the ventilators are pair of circular holes provided at the top component of the said device.

8. (Previously presented) A see through testing device as claimed in claim 7, wherein the ventilators are pair of slits provided at the center of the top component of the said device.

9. (Previously presented) A see through testing device as claimed in claim 7, wherein the diameter of the pair of said ventilators is 10 mm².

10. (Previously presented) A see through testing device as claimed in claim 1, wherein the top component and bottom receptacle are rectangular in shape.

11. (Previously presented) A see through testing device as claimed in claim 1, wherein the sample cup is rectangular in shape having the width at its bottom of 2 mm and depth of 2.5 mm to 3.0 mm.

12. (Previously presented) A see through testing device as claimed in claim 1, wherein length of the tracks provided adjacent to the sink pad of the test strip is more than the length of the tracks adjacent to the sample pad of the test strip.

13. (New) A see through testing device as claimed in claim 5, wherein the bars are positioned on the inner side of the top component.
